(19) United States
(12) Patent Application Publication
(10) Pub. No.: US 2009/0035816 A1
(43) Pub. Date: Feb. 5, 2009

(54) Process for the preparation of a polypeptide

(75) Inventors: Wai Hong Chan, San Mateo, CA (US); Jin Guo Ding, Shanghai (CN); Mei Hua Xie, Shanghai (CN); Tsung Yu Hsiao, Kaohsiung County (CN)

Correspondence Address: COHEN, PONTANI, LIEBERMAN & PAVANE LLP, 551 FIFTH AVENUE, SUITE 1210, NEW YORK, NY, 10176, US

(73) Assignee: ScinoPharm Taiwan Ltd., Tainan County (TW)

(21) Appl. No.: 12/221,092

(22) Filed: Jul. 31, 2008

Related U.S. Application Data
(60) Provisional application No. 60/963,027, filed on Aug. 2, 2007.

(51) Int. Cl. C12P 21/06; C07K 1/06

(52) U.S. Cl. 435/68.1; 530/333

(57) ABSTRACT

A process for the preparation of a polypeptide made from amino acids L-alanine, L-glutamic acid, L-lysine, and L-tyrosine comprising using N-thiocarboxyanhydride of at least one amino acid as a starting material.

### RELATED APPLICATIONS

This application claims priority from U.S. Provisional Patent Application Ser. No. 60/963,027 which was filed on Aug. 2, 2007. The entire content of U.S. Provisional Patent Application Ser. No. 60/963,027 is incorporated herein by reference.

### BACKGROUND OF THE INVENTION

1. Field of the Invention

The present invention relates to an improved process for the preparation of a polypeptide or pharmaceutically acceptable salt thereof and intermediates useful in the synthesis thereof.

2. Description of the Related Art

The present invention relates to a new process for the synthesis of polypeptides comprising the following amino acid units in the structure, namely,: L-alanine, L-glutamic acid, L-lysine, and L-tyrosine. Glatiramer acetate, also known as copolymer-1, is a representative polypeptide of the present invention.

Glatiramer acetate is a mixture of polypeptides which has been approved for the treatment of multiple sclerosis. It is a mixture of acetate salts of chemically synthetic polypeptides, containing four naturally occurring amino acids: L-alanine, L-glutamic acid, L-lysine, and L-tyrosine with an average molar ratio of 0.427, 0.141, 0.338, and 0.095, respectively. The average molecular weight of glatiramer acetate is 4,700-11,000 daltons.

Chemically, glatiramer acetate is designated L-glutamic acid polymer with L-alanine, L-lysine and L-tyrosine, acetate (salt). Its structural formula is: (Glu, Ala, Lys, Tyr)ₓ.xCH₃COOH. Its CAS number is 147245-9-2-9.

Processes for preparing polypeptides of this type, including glatiramer acetate have been described in U.S. Pat. No. 3,849,550; U.S. Patent Publication Nos. 2006/0172942 and 2006/0154862. The entire content of these patents and patent publications is incorporated herein as reference. The process for the preparation of the polypeptides of this type is based on the copolymerization of N-carboxyanhydride of tyrosine, N-carboxyanhydride of L-alanine, N-carboxyanhydride of protected L-glutamic acid and N-carboxyanhydride of protected L-lysine to form a protected copolymer. The deblocking of the protected L-glutamic acid is effected by acidolysis or hydrogenolysis (first deprotection) and is followed by the removal of the protecting group from L-lysine by base cleavage (second deprotection).

U.S. Pat. No. 7,049,399, which is incorporated herein as reference, describes a process for preparing a polypeptide comprising a single step deprotection of a protected copolymer, which is formed by copolymerizing of N-carboxyanhydride of tyrosine, N-carboxyanhydride of L-alanine, N-carboxyanhydride of protected L-glutamic acid and N-carboxyanhydride of protected L-lysine. The deblocking of all the protecting groups is effected by a single reaction step such as hydrogenolysis.

The references discussed above all disclose the use of N-carboxyanhydrides of the four amino acids as starting materials to obtain protected polypeptides, including galtiramer acetate. Nevertheless, N-carboxyanhydrides of the four amino acids exhibit poor stability and therefore cannot ensure production of polypeptides with consistently desirable molecular weight and ratio of different amino acids.

Therefore, improvement of production of such polypeptides is desirable.

### SUMMARY OF THE INVENTION

The new and improved process, according to the present invention, is based on the use of N-thiocarboxyanhydride of amino acid (see formula 1 shown below) instead of N-carboxyanhydride of amino acid (see formula 2 shown below) as the starting material in copolymerization:

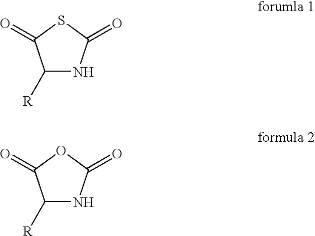

R represents the side chain of L-alanine, L-tyrosine that is optionally protected, protected L-glutamate, or protected L-lysine.

Specifically, the present invention is directed to a new process for the preparation of a polylpeptide or a pharmaceutically acceptable salt thereof. The polypeptide comprises L-alanine, L-glutamic acid, L-lysine, and L-tyrosine. The process comprises a) polymerizing L-alanine, L-tyrosine that is optionally protected, protected L-glutamate, and protected L-lysine in a solvent to produce a protected polypeptide; and b) deprotecting the protected polypeptide to obtain the polylpeptide or a pharmaceutically acceptable salt thereof. At least one of L-alanine, L-tyrosine that is optionally protected, protected L-glutamate, and protected L-lysine is in the form of N-thiocarboxyanhydride as shown in formula 1.

In accordance with one preferred embodiment of the present invention, at least L-alanine used in the polymerizing step is in form of N-thiocarboxyanhydride as shown in formula 1.

Preferably, the polymerizing step is carried out in the presence of an inert solvent. For example, the inert solvent can be selected from the group consisting of, dimethylsulfone, dioxane, dimethylformamide, dichloromethane, tetrahydrofuran, N-methylpyrrolidone, sulfolane, nitrobenzene, tetramethylurea, and mixtures thereof.

As a preferred embodiment, the polymerizing step is carried out in the presence of an initiator. The initiator is preferably selected from the group consisting of diethylamine, triethylamine, diisopropyalmine, hexylamine, phenethylamine, sodium methoxide, sodium t-butoxide, transition metal initiator bbyNi(COD), (Pme3)4Co, and mixtures thereof.

As a preferred embodiment, the polymerization of the present invention is normally carried out at room temperature, 1 atmospheric pressure for about 48 hours.

In accordance with one preferred embodiment of the present invention, L-tyrosine is protected by, for example, an organic group which can be removed by base cleavage, acidolysis, thiolysis, hydrogenation or enzyme-catalyzed hydrolysis. The organic protecting group can be an alkyl group of more than three carbon atoms and/or aromatic group. More preferably, the protecting group is selected from benzyl, 2,6-dichlorobenzyl, 2-bromobenzyloxycarbonyl, t-butyl, and 2,4-dinitrophenyl. U.S. Patent Publication No. 2007/0141663 discloses process of making a polypeptide comprising: a) polymerizing L-alanine, protected L-tyrosine, protected L-glutamate, and protected L-lysine to obtain a protected polypeptide; and b) deprotecting the protected polypeptide in one step by base cleavage, acidolysis, thiolysis, hydrogenation, or enzyme-catalyzed hydrolysis to produce the polypeptide. The content of the entire U.S. Patent Publication No. 2007/0141663 is incorporated herein as reference.

In accordance with a preferred embodiment of the present invention, the lysine may be protected by an alkyl group of more than three carbon atoms and/or an aromatic group, more preferably, by a group selected from benzyloxycarbonyl, 4-methoxybenzyloxycarbonyl, α,α-dimethyl 3,5-dimethoxybenzyloxy, 2-(4-biphenylyl)isopropoxycarbonyl, t-butyloxycarbonyl, 2,2,2-trichloroethoxycarbonyl, t-amyloxycarbonyl, adamantyloxycarbonyl, allyloxycarbonyl, o-nitrophenylsulfenyl, trityl, 9-fluorenylmethyloxycarbonyl, phenylacetyl, pyroglutamyl, and combinations thereof.

In accordance with another preferred embodiment of the present invention, glutamic acid can be protected by an alkyl group of more than three carbon atoms and/or an aromatic group, more preferably, by a group selected from cyclohexyl ester, benzyl ester, t-butyl ester, allyl ester, adamantyl, 9-fluorenylmethyl, and combinations thereof.

The deprotecting step can be accomplished by, for example, base cleavage, acidolysis, thiolysis, hydrogenation, or enzyme-catalyzed hydrolysis.

In accordance with one embodiment of the present invention, the deprotecting step comprises adding an acid to the protected polypeptide. The acid can be, for example, hydrobromide, trifluoroacetic acid, or hydrogen chloride in a solvent medium selected from acetic acid, dioxane, ethyl acetate, and mixtures thereof. As a preferred embodiment, the acid can be 40% hydrobromic acid dissolved in acetic acid.

In accordance with another embodiment of the present invention, the deprotecting step comprises 1) removing the protected group from L-glutamic acid by an alkali; and 2) removing the protected group from L-lysine by an acid. The alkali can be, for example, dimethyl formamide, sodium hydroxide, piperidine. As a preferred embodiment, the alkalin can be aqueous NaOH. The acid can be, for example, 40% hydrobromic acid dissolved in acetic acid.

Subsequent to the deprotecting step, the polypeptide can be further isolated or purified. For example, the isolation and purification can be carried out in a single step by a single dialysis against water.

As one of the preferred embodiments, the polypeptide obtained in the present invention is glatiramer acetate.

In accordance yet with another embodiment, the present application provides a new polypeptide produced in accordance with the process described above.

Compared to the process disclosed in other references, N-thiocarboxyanhydride of amino acids used in the present invention, in particular N-thiocarboxyanhydride of L-alaine, is far more chemically stable than the corresponding N-carboxyanhydride of amino acid (J. Org. Chem. 1971, 36, 49-59). As a monomer, the four amino acids involved in the present invention, in particular L-alaine, if existing in form of N-carboxyanhydride, is very unstable. Therefore, it is very difficult to store or control the quality of the N-carboxyanhydride form of the four amino acids involved in the present invention. On the other hand, due to the superior stability of N-thiocarboxyanhydride of the same four amino acids involved in the present invention, the use of N-thiocarboxyanhydride of amino acids results in ease of storage and transportation of starting materials, simplicity in polymerizing reaction, and a cost-effective process. The use of N-thiocarboxyanhydride in producing polypeptide made from L-alanine, L-glutamic acid, L-lysine, and L-tyrosine also significantly improves the consistency of the molecular weight and ratio of different amino acids in the final polypeptide product.

The various features of novelty which characterize the invention are pointed out with particularity in the claims annexed to and forming a part of the disclosure. For a better understanding of the invention, its operating advantages, and specific objects attained by its use, reference should be had to the descriptive matter in which there are illustrated and described preferred embodiments of the invention.

### DETAILED DESCRIPTION OF THE PRESENTLY PREFERRED EMBODIMENTS

In accordance with one embodiment of the present invention, the process for the preparation of a polypeptide composed of L-alanine, L-glutamic acid, L-lysine, and L-tyrosine, or a pharmaceutically acceptable salt thereof, comprises the steps of:
a. Polymerizing a mixture of N-thiocarboxyanhydride of L-alanine, N-carboxyanhydride of L-tyrosine, N-carboxyanhydride of protected L-glutamate and N-carboxyanhydride of protected L-lysine to obtain a protected polypeptide;
b. Deprotecting the protected polypeptide to afford the polypeptide by an acid or a pharmaceutically salt thereof; and
c. Isolating and/or purifying the polypeptide or a pharmaceutically acceptable salt thereof.

In one embodiment, the process comprises polymerizing N-thiocarboxyanhydride of L-alanine, N-carboxyanhydride of gama-benzyl L-glutamate, N-carboxyanhydride of e-N-benzyloxycarbonyl L-lysine, and N-carboxyanhydride of L-tyrosine in an inert solvent with a initiator. After the completion of the polymerization, water was added to the reaction mixture to precipitate the fully protected polypeptide. All the protecting groups on the corresponding protected polypeptide can be removed by hydrogen bromide in glacial acetic acid. Upon the completion of the de-protection, deprotected group, excess hydrobromic acid and acetic acid were removed to give a crude polypeptide. The crude polypeptide was purified by Sephadex G50 eluting with 1 N acetic acid and collecting the polypeptide acetic salt with the desired molecular weight range.

In accordance with another embodiment of the present invention, the process for the preparation of a polypeptide composed of L-alanine, L-glutamic acid, L-lysine, and L-tyrosine, or a pharmaceutically acceptable salt thereof, comprises the steps of:
a. polymerizing of a mixture of N-thiocarboxyanhydride of L-alanine, N-carboxyanhydride of L-tyrosine, N-carboxyanhydride of protected L-glutamate and N-carboxyanhydride of protected L-lysine in a solvent to obtain a protected polypeptide;
b. removing the protected group from L-glutamic acid by an alkali;
c. removing the protected group from L-lysine chain by an acid to obtain the polypeptide or a pharmaceutically acceptable salt thereof; and
d. isolating and/or purifying the polypeptide or a pharmaceutically acceptable salt thereof.

In one embodiment, the process comprises polymerizing N-thiocarboxyanhydride of L-alanine, N-carboxyanhydride of gama-benzyl L-glutamate, N-carboxyanhydride of e-N-benzyloxycarbonyl L-lysine, and N-carboxyanhydride of L-tyrosine in an inert solvent with a initiator. After the completion of the polymerization, water was added to the reaction mixture to precipitate the fully protected polypeptide. Gama-benzyl protecting group on L-glutamic acid was removed by an alkali such as dimethyl formamide/NaOH/water. The e-N-Benzyloxycarbonyl protected polypeptide was precipitated by neutralization and then treated by hydrogen bromide in glacial acetic acid to remove the protecting group. Upon the completion of the de-protection, deprotected group, excess hydrobromic acid and acetic acid was removed to give a crude polypeptide. The crude polypeptide was purified by Sephadex G50 eluting with 1 N acetic acid with collecting the copolymer-1 acetic salt with the desired molecular weight range.

In accordance with yet another embodiment of the present invention, the process of making a protected polypeptide comprises polymerizing a mixture of four amino acids derivatives, L-alanine, L-tyrosine, protected L-glutamate and protected L-lysine. At least one of the four amino acid derivatives is an N-thiocarboxyanhydride.

In an embodiment, the process comprise polymerizing N-thiocarboxyanhydride of L-alanine, N-carboxyanhydride of gama-benzyl L-glutamate, N-carboxyanhydride of e-N-benzyloxycarbonyl L-lysine, and N-carboxyanhydride of L-tyrosine in an inert solvent with a initiator. After the completion of the polymerization, water was added to the reaction mixture to precipitate the fully protected copolymer-1.

The following examples are provided for further illustration, but not for limitation, of the present invention.

### EXAMPLE 1

### Fully Protected Copolymer-1 Preparation

0.166 g of N-thiocarboxyanhydride of L-alanine, 0.111 g of N-carboxyanhydride of gama-benzyl L-glutamate, 0.303 g of N-carboxyanhydride of e-N-benzyloxycarbonyl L-lysine, and 0.060 g of N-carboxyanhydride of L-tyrosine were dissolved in 8.1 ml of dioxane to which 3.3 ml of diethyl amine in dioxane (0.5 g/L) was added. The reaction mixture was stirred at room temperature for 48 hours. The reaction mixture was poured into 50 ml of water with good agitation. The white precipitation was filtered and washed subsequently with water and acetone. After drying in vacuum, 0.427 g (85.4% yield based on the total weight) of fully protected polypeptide was obtained.

### EXAMPLE 2

### Copolymer-1 Preparation by Acidolysis

0.200 g of protected polypeptide obtained by the method described in Example 1) was added to 10 ml of 40% hydrobromic acid dissolved in acetic acid and stirred at 30° C. for 16 hours. 0.181 g of crude product was precipitated from the reaction mixture by adding 30 ml of ethyl ether. 0.050 g of crude product was dissolved in 1 ml of 1 N aqueous acetic acid and then was loaded on a Sephadex G50 (2.8×32 cm) column which was equilibrated and eluted with 1 N acetic acid. The elution between 58˜105 ml was collected and dried in vacuum to give 20.2 mg of copolymer-1 acetate with a yield of 47% calculated based on the weight of starting material.

### EXAMPLE 3

### Copolymer-1 Preparation by Combination of Base Cleavage with Acidolysis

0.200 g of protected polypeptide obtained by the method described in Example 1 was dissolved in 3 ml of dimethyl formamide and 0.25 ml of 5 N aqueous NaOH solution was added. After being stirred at 25° C. for 2 hours, the reaction solution was neutralized to pH 7 by adding 3 ml of 1 N aqueous HCl solution in ice bath and then diluted with 10 ml of water to obtain 0.176 mg of precipitant. All the dried precipitant was added to 5 ml of 40% hydrobromic acid dissolved in acetic acid and stirred at 30° C. for 4 hours. 0.182 g of crude product was precipitated from the reaction mixture by adding 30 ml of ethyl ether). 0.090 g of crude product was dissolved in 1 ml of 1 N aqueous acetic acid and then was loaded on a Sephadex G50 (2.8×32 cm) column which was equilibrated and eluted with 1 N acetic acid. The elution between 58˜105 ml was collected and dried in vacuum to give 16.1 mg of copolymer-1 acetate with a yield of 21% calculated based on the weight of starting material.

The invention is not limited by the embodiments described above which are presented as examples only but can be modified in various ways within the scope of protection defined by the appended patent claims.

We claim:

1. A process of making a polylpeptide or a pharmaceutically acceptable salt thereof comprising:
a) polymerizing L-alanine, L-tyrosine that is optionally protected, protected L-glutamate, and protected L-lysine in a solvent to obtain a protected polypeptide; and
b) deprotecting the protected polypeptide to obtain the polylpeptide or a pharmaceutically acceptable salt thereof;
wherein at least one of L-alanine, L-tyrosine that is optionally protected, protected L-glutamate, and protected L-lysine is an N-thiocarboxyanhydride as shown in formula 1 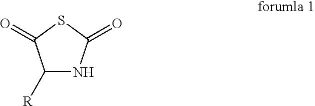
R represents the side chain of L-alanine, L-tyrosine that is optionally protected, protected L-glutamate, and protected L-lysine.

2. The process of claim 1 wherein L-alanine used in the polymerizing step is an N-thiocarboxyanhydride.

3. The process of claim 1 wherein the solvent used in the polymerizing step is selected from the group consisting of DMF, DMSO, CH₂Cl₂, dioxane or mixtures thereof.

4. The process of claim 1 wherein the polymerizing step is carried out in the presence of an initiator selected from the group consisting of diethylamine, triethylamine, diisopropyalmine, and mixtures thereof.

5. The process of claim 1 wherein L-tyrosine used in the polymerizing step is protected, and the protecting groups of L-tryosine, L-glutamate, and L-lysine are deprotected in the same step.

6. The process of claim 1 wherein the deprotecting step is accomplished by a method selected from the group consisting of base cleavage, acidolysis, thiolysis, hydrogenation, enzyme-catalyzed hydrolysis, and combinations thereof.

7. The process of claim 6 wherein the deprotecting step is accomplished by the acidolysis method in one step.

8. The process of claim 6 wherein the deprotecting step is accomplished by the combination of base cleavage and acidolysis, and the deprotecting step comprises 1) removing the protecting group from L-glutamic acid by an alkali; and 2) removing the protecting group from L-lysine by an acid.

9. The process of claim 1 further comprising, subsequent to the deprotecting step, an isolating and/or purifying step to isolate or purify the polypeptide.

10. The process of claim 1 wherein each of the L-tyrosine that is optionally protected, protected L-glutamate, and protected L-lysine is an N-carbonanhydride of formula 2: 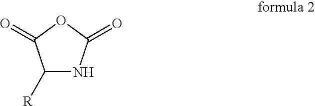
wherein R represents the side chain of L-tyrosine that is optionally protected, protected L-glutamate, and protected L-lysine.

11. The process of claim 1 wherein the protected L-glutamate is N-carboxyanhydride of gama-benzyl L-glutamate, and the protected L-lysine is N-carboxyanhydride of e-N-benzyloxycarbonyl L-lysine.

12. The process of claim 1 wherein the polypeptide is glatiramer acetate.

13. A polypeptide or a pharmaceutically acceptable salt thereof, comprising L-alanine, L-tyrosine, L-glutamate, and L-lysine, prepared according to a process comprising the steps:
a) polymerizing L-alanine, L-tyrosine that is optionally protected, protected L-glutamate, and protected L-lysine to obtain a protected polypeptide; and
b) deprotecting the protected polypeptide to obtain the polylpeptide or a pharmaceutically acceptable salt thereof;
wherein at least one of L-alanine, L-tyrosine that is optionally protected, protected L-glutamate, and protected L-lysine is an N-thiocarboxyanhydride as shown in formula 1 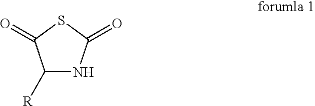
R represents the side chain of L-alanine, L-tyrosine that is optionally protected, protected L-glutamate, and protected L-lysine.

14. A process of making a polylpeptide or a pharmaceutically acceptable salt thereof comprising:
a) polymerizing L-alanine, L-tyrosine that is optionally protected, protected L-glutamate, and protected L-lysine in a solvent to obtain a protected polypeptide; and
b) deprotecting the protected polypeptide to obtain the polylpeptide or a pharmaceutically acceptable salt thereof;
wherein the deprotecting step is accomplished by the combination of base cleavage and acidolysis, and the deprotecting step comprises 1) deprotecting the protected L-glutamate by an alkali; and 2) deprotecting the protected L-lysine by an acid.

15. The process of claim 14 wherein at least one of L-alanine, L-tyrosine that is optionally protected, protected L-glutamate, and protected L-lysine is an N-thiocarboxyanhydride as shown in formula 2 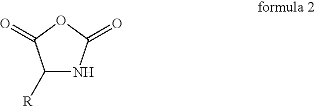
R represents the side chain of L-alanine, L-tyrosine that is optionally protected, protected L-glutamate, and protected L-lysine.
